(19) United States
(12) Patent Application Publication
(10) Pub. No.: US 2023/0113324 A1
(43) Pub. Date: Apr. 13, 2023

(54) MEDICAL INFORMATION PROCESSING DEVICE, MEDICAL INFORMATION PROCESSING METHOD, AND STORAGE MEDIUM

(71) Applicant: CANON MEDICAL SYSTEMS CORPORATION, Otawara-shi (JP)

(72) Inventors: Kosuke ARITA, Otawara (JP); Longxun PIAO, Nasushiobara (JP); Sho SASAKI, Utsunomiya (JP); Yudai YAMAZAKI, Nasushiobara (JP)

(73) Assignee: CANON MEDICAL SYSTEMS CORPORATION, Otawara-shi (JP)

(21) Appl. No.: 17/937,863

(22) Filed: Oct. 4, 2022

(30) Foreign Application Priority Data
Oct. 13, 2021 (JP) 2021-168101

(51) Int. Cl. A61B 5/00; G16H 50/30; G16H 50/70

(52) U.S. Cl. CPC A61B 5/7275; G16H 50/30; G16H 50/70

(57) ABSTRACT

A medical information processing device according to an embodiment includes processing circuitry. The processing circuitry is configured to acquire biometric information on a patient, detect a trend change in the biometric information at a predetermined timing on a basis of time-series data regarding the biometric information, and determine whether the trend change is due to a measurement error in the biometric information or due to a change in a condition of the patient.

### CROSS-REFERENCE TO RELATED APPLICATION

The present application claims priority based on Japanese Patent Application No. 2021-168101 filed Oct. 13, 2021, the content of which is incorporated herein by reference.

### FIELD

Embodiments disclosed in the present specification and drawings relate to a medical information processing device, a medical information processing method, and a storage medium.

### BACKGROUND

In order to accurately and rapidly ascertain changes in physical condition, continuous home monitoring based on easily measurable patient information (e.g., a blood pressure, a percutaneous arterial blood oxygen saturation value, a body temperature, pulse measured from facial images, etc.) is desirable. However, since a patient who is not a medical professional performs measurement himself at home, various measurement abnormalities such as misalignment of a mounting position and body movement are likely to occur. When monitoring is performed using information including such measurement abnormalities, it is likely to lead to errors in interpretation of measurement results, making it difficult to accurately ascertain physical condition.

Accordingly, for example, a health management system that detects measurement of unusual vitals due to drinking alcohol, lack of sleep, or the like and determines that vital fluctuations are not caused by a disease has been proposed. Further, a technique for determining an abnormality in a measurement environment on the basis of an abnormality in correlation between measured values of a patient has been proposed.

### BRIEF DESCRIPTION OF THE DRAWINGS

FIG. 1 is a diagram showing an example of a configuration of a medical information processing device of an embodiment.

FIG. 2 is a diagram showing an example of information stored in a patient DB.

FIG. 3 is a diagram showing an example of identifying similar patients from the patient DB.

FIG. 4 is a diagram showing an example of a trend score calculation method performed by a first calculation function and a second calculation function.

FIG. 5 is a diagram showing an example of trend scores calculated by the first calculation function and the second calculation function.

FIG. 6 is a diagram showing a trend score calculation method when a plurality of similar patient conditions are extracted from the patient DB.

FIG. 7 is a diagram showing a method of obtaining a trend score according to principal component analysis.

FIG. 8 is a diagram showing a method of obtaining a trend score according to maximum likelihood estimation.

FIG. 9 is a diagram showing processing performed by a determination function.

FIG. 10 is a flowchart showing an example of a trend score calculation and determination processing procedure.

FIG. 11 is a diagram showing an example of a first trend score and an example of a second trend score for each case.

FIG. 12 is a flowchart of an example of a processing procedure performed by the medical information processing device according to an embodiment.

FIG. 13 is a diagram showing an example of a usage scenario of the medical information processing device.

FIG. 14 is a diagram showing an example of information presented when it is determined that there is no measurement error and no disease onset.

FIG. 15 is a diagram showing an example of information presented when it is determined that there is a measurement error and that there is no disease onset.

FIG. 16 is a diagram showing an example of information presented when it is determined that there is disease onset.

FIG. 17 is a diagram showing an example of a configuration of a medical information processing device of a modified example.

FIG. 18 is a diagram showing an example of an image presented on a presentation device.

### DETAILED DESCRIPTION

Hereinafter, a medical information processing device, a medical information processing method, and a storage medium according to embodiments will be described with reference to the drawings.

A medical information processing device according to an embodiment includes processing circuitry. The processing circuitry is configured to acquire biometric information on a patient, detect a trend change in the biometric information at a predetermined timing on a basis of time-series data regarding the biometric information, and determine whether the trend change is due to a measurement error in the biometric information or due to a change in a condition of the patient. According to the medical information processing device of the embodiment, it is possible to appropriately determine whether or not values measured by a patient or the like are normal.

### Configuration of Medical Information Processing Device

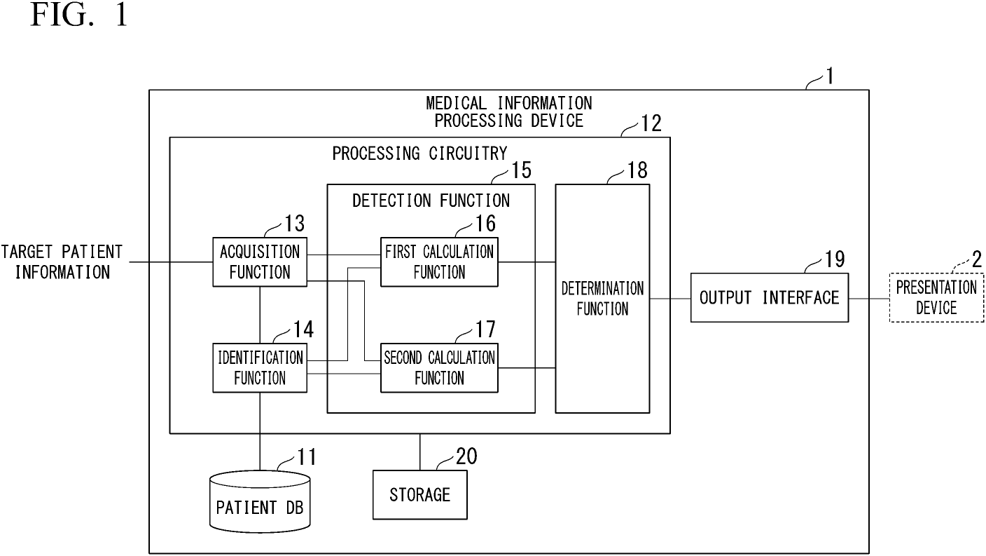

FIG. 1 is a diagram showing an example of a configuration of a medical information processing device according to the present embodiment. As shown in FIG. 1, the medical information processing device 1 includes a patient database (DB) 11, processing circuitry 12, an output interface 19, and a storage 20. The processing circuitry 12 includes an acquisition function 13, an identification function 14, a detection function 15, and a determination function 18. The detection function 15 includes a first calculation function 16 and a second calculation function 17.

The medical information processing device 1 outputs, to a presentation device 2, presentation information that is results in which abnormal measurement, normal physical condition, and disease onset are distinguished on the basis of closeness of trend between acquired biometric information on a target patient and biometric information on other patients with reference to information stored in the patient DB 11. The presentation device 2 is connected to the medical information processing device 1.

The presentation device 2 is, for example, at least one of an image display device, a printing device, a smartphone, a tablet terminal, and the like. The presentation device 2 presents presentation information output by the medical information processing device 1.

The patient DB 11 stores patient information on each of a plurality of patients other than the target patient (hereinafter simply referred to as a “patient”). Patient information includes, for example, patient identification information, patient disease information, patient age information, patient sex information, patient biometric information (measurement value information), and the like. The patient biometric information includes, for example, a blood pressure value, a pulse rate, a respiratory rate, a percutaneous arterial blood oxygen saturation value, and the like. All or part of the patient information of a patient may be stored in the patient DB 11.

The acquisition function 13 acquires patient information of a patient which will be determined. The patient information includes, for example, patient biometric information and patient attribute information. The patient biometric information includes, for example, a blood pressure, a pulse rate, a respiratory rate, a percutaneous arterial oxygen saturation (SpO₂) value, and the like. The patient attribute information is, for example, information such as age, sex, and disease history. The acquisition function 13 continuously acquires the patient biometric information, for example, at each of sampling times. The sampling times are, for example, every second, every minute, every hour, every day, morning, afternoon or evening, or the like. Moreover, there are two or more types of biometric information. If attribute information among the patient information is stored in the patient DB 11, the acquisition function 13 may acquire the attribute information from the patient DB 11. The acquisition function 13 outputs the acquired patient information of the patient to the identification function 14, the first calculation function 16, and the second calculation function 17.

The identification function 14 identifies a similar patient who is close to attributes of the patent from the patient DB 11 with respect to the patient attribute information output by the acquisition function 13 and extracts or calculates a measured value distribution of the identified similar patient. A similar patient is a patient whose attributes are closest to the target patient in the same disease patient population, for example, a patient who is of the same sex and the same age or is within a predetermined range thereof. Further, when a plurality of similar patients are extracted, the identification function 14 extracts a measurement value distribution for each similar patient. The identification function 14 calculates the measured value distribution of the similar patients by calculating, for example, a probability distribution for a plurality of measured values of the similar patient extracted from the patient DB 11. Therefore, the measured value distribution is, for example, data stored in the patient DB 11, a probability distribution calculated from the data, and the like. The identification function 14 outputs measured values when the similar patient is healthy and when the similar patient is diseased.

The detection function 15 detects a trend change in the biometric information at a predetermined timing on the basis of time-series data with respect to the biometric information.

The first calculation function 16 receives the patient biometric information obtained by the acquisition function 13 and a measured value distribution (biometric information on the similar patient) of the similar patient in a certain healthy state (one or more types) identified by the identification function 14. The first calculation function 16 calculates a first trend score representing closeness of trend between the patient biometric information and the measured value distribution of similar patient information in the healthy state. The first calculation function 16 outputs the calculated first trend score to the determination function 18.

The second calculation function 17 receives the patient biometric information acquired by the acquisition function 13 and a measured value distribution of the similar patient in a certain state (one or more types) at the time of disease onset identified by the identification function 14. The second calculation function 17 calculates a second trend score representing closeness of trend between the patent biometric information and the measured value distribution of the similar patient in a diseased state. The second calculation function 17 outputs the calculated second trend score to the determination function 18.

The determination function 18 compares the first trend score output by the first calculation function 16 with the second trend score output by the second calculation function 17 and outputs the comparison result to the output interface 19.

The output interface 19 outputs the comparison result output by the determination function 18 to the presentation device 2. The output interface 19 may have a wired or wireless communication function.

The storage 20 stores thresholds, formulas, programs for performing processing, and the like used in processing of the medical information processing device 1.

The example of the configuration shown in FIG. 1 is an example, and is not limited thereto. For example, the patient DB 11 may be connected to the medical information processing device 1 via a network. Further, the medical information processing device 1 may also include an input interface to which input devices such as a keyboard and a mouse are connected.

The input interface in this description is not limited to those having physical operation parts such as a mouse and a keyboard. For example, examples of the input interface also include electrical signal processing circuitry that receive an electrical signal corresponding to an input operation from an external input apparatus provided separately from the device and output the electrical signal to control circuitry.

In addition, the processing circuitry 12 realizes the functions of the acquisition function 13, the identification function 14, the detection function 15, the first calculation function 16, the second calculation function 17, and the determination function 18, for example, by a hardware processor executing a program stored in the storage 20.

The hardware processor refers to, for example, circuitry such as a central processing unit (CPU), a graphics processing unit (GPU), an application specific integrated circuit (ASIC), or a programmable logic device (for example, a simple programmable logic device (SPLD), a complex programmable logic device (CPLD), or a field programmable gate array (FPGA)). Instead of being stored in the storage 20, the program may be directly incorporated into the circuitry of the hardware processor. In this case, the hardware processor realizes functions by reading and executing the program incorporated in the circuitry. The hardware processor is not limited to being configured as single circuitry and may be configured as one hardware processor by combining a plurality of independent circuitry to realize each function. Further, a plurality of components may be integrated into one hardware processor to realize each function.

### Example of Information Stored in Patient DB

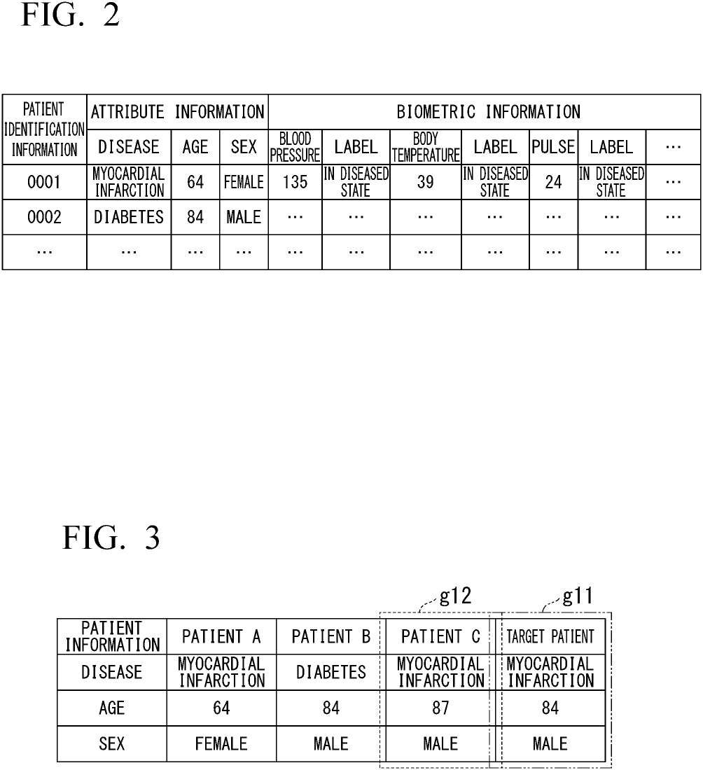

Next, an example of information stored in the patient DB 11 will be described. FIG. 2 is a diagram showing an example of information stored in the patient DB. As shown in FIG. 2, the patient DB 11 stores, for example, patient identification information in association with attribute information and measured values. The attribute information is, for example, a disease name, age, sex, and the like, as described above. The measured values are, for example, a blood pressure, a body temperature, and pulse, each of which is associated with a label indicating a healthy state or a diseased state. The example shown in FIG. 2 is an example, and it is not limited thereto.

### Processing Performed by Identification Function

Next, processing performed by the identification function 14 will be further described. The identification function 14 identifies a similar patient from the patient DB 11 on the basis of attribute information on a target patient and extracts a measured value distribution of the identified similar patient. If there is no patient with the same disease, the identification function 14 may extract a patient whose attribute information is closest or the target patient himself/herself.

FIG. 3 is a diagram showing an example of identifying a similar patient from the patient DB. As indicated by a dashed line g11 in FIG. 3, patient attribute information represents that the disease is myocardial infarction, the age is 84 years old, and the sex is male. In this case, the identification function 14 identifies, from the patient DB 11, a patient C who has the same underlying disease and who is of the same sex as the target patient and an age close to the target patient, as indicated by a dashed line g12 in FIG. 3. In this manner, the identification function 14 identifies at least one patient whose attributes are close to the patient from the patient DB 13. If no patient with the same disease is stored in the patient DB 11, the identification function 14 may identify a patient who is of the same sex and is within a predetermined age range.

Here, measured values (biometric information) will be described.

It is desirable that measured values stored in the patient DB 11 be measured values acquired in a situation in which the probability of measurement errors is low (for example, during hospitalization or outpatient treatment). In addition, the measured values stored in the patient DB 11 may be values obtained by performing preprocessing (for example, smoothing) for reducing measurement errors on measured values in order to curb the influence of measurement errors. Alternatively, the identification function 14 may perform preprocessing on the measured values stored in the patient DB 11 during use. In addition, the measured values stored in the patient DB 11 are labeled with, for example, the physical condition at the time of measurement. The label is, for example, label 0 in case of a healthy condition (in case of no disease onset) and label 1 in case of disease onset.

### Example of Score Calculation Method

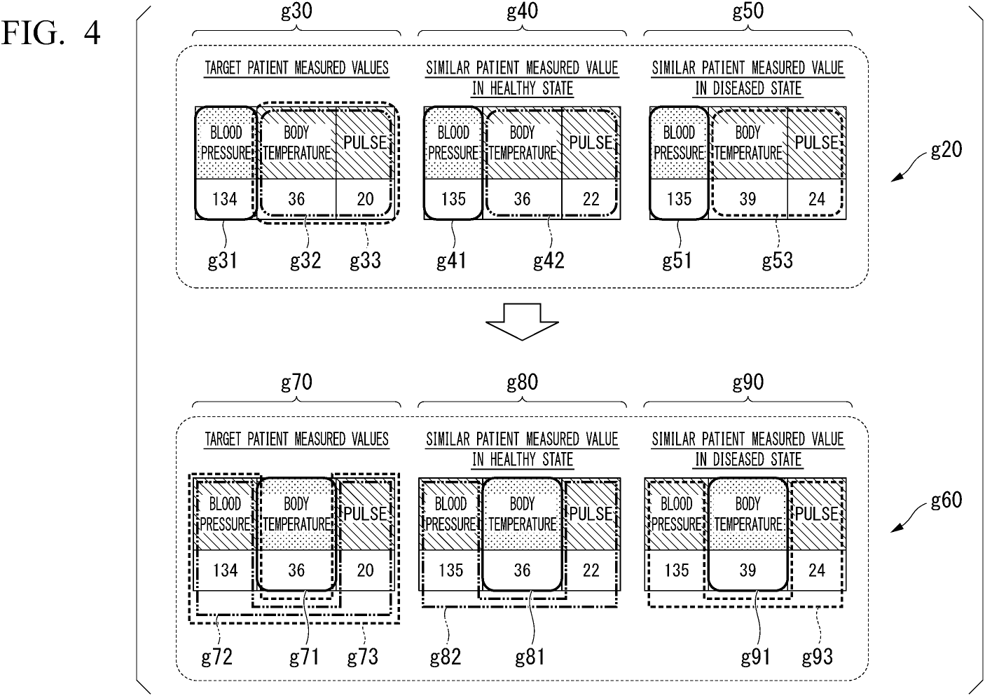

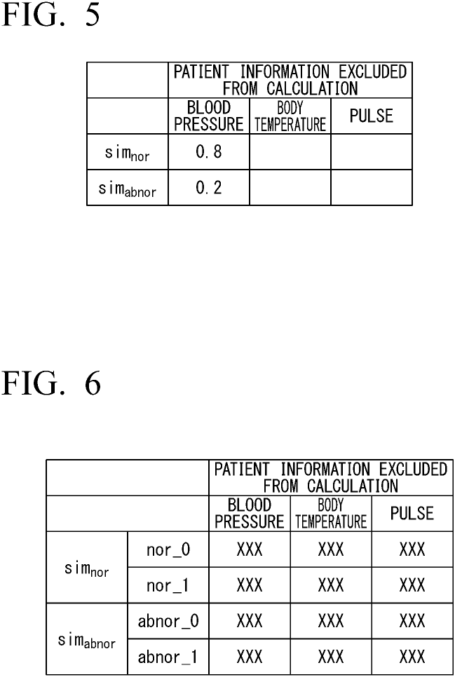

Next, an example of a trend score calculation method performed by the first calculation function 16 and the second calculation function 17 will be described with reference to FIG. 4 and FIG. 5. FIG. 4 is a diagram showing an example of a trend score calculation method performed by the first calculation function and the second calculation function. FIG. 5 is a diagram showing an example of trend scores calculated by the first calculation function and the second calculation function. In the examples shown in FIGS. 4 and 5, measured values are blood pressure, body temperature, and pulse.

As in an image g20 in FIG. 4, the first calculation function 16 and the second calculation function 17 exclude blood pressures (g31, g41, and g51) from measured values (biometric information) (g30) of a patient, measured values (biometric information) (g40) of a similar patient in a healthy state, and measured values (biometric information) (g50) of the similar patient in a diseased state. The first calculation function 16 calculates a first trend score Simₙₒᵣ between the temperature and pulse (g32) of the patient and the temperature and pulse (g42) of the similar patient in a healthy state. As a result of calculation, the first trend score Simₙₒᵣ is 0.8 as shown in FIG. 5. The second calculation function 17 calculates a second trend score Sim_{abnor} between the temperature and pulse (g33) of the patient and the temperature and pulse (g53) of the similar patient in a diseased state. As a result of calculation, the second trend score Sim_{abnor} is 0.2 as shown in FIG. 5.

Next, as in an image g60 in FIG. 4, the first calculation function 16 and the second calculation function 17 exclude body temperatures (g71, g81, and g91) from measured values (biometric information) (g70) of the patient, measured values (biometric information) (g80) of the similar patient in a healthy state, and measured values (biometric information) (g90) of the similar patient in a diseased state. The first calculation function 16 calculates a first trend score Siniₙₒᵣ between the blood pressure and pulse (g72) of the patient and the blood pressure and pulse (g82) of the similar patient in a healthy state. The second calculation function 17 calculates a second trend score Sim_{abnor} between the blood pressure and pulse (g73) of the target patient and the blood pressure and pulse (g93) of the similar patient in a diseased state.

Thereafter, the first calculation function 16 and the second calculation function 17 exclude pulses from measured values of the patient, measured values of the similar patient in a healthy state, and measured values of the similar patient in a diseased state. The first calculation function 16 calculates a first trend score Simₙₒᵣ between the blood pressure and the body temperature of the patient and the blood pressure and the body temperature of the similar patient in a healthy state. The second calculation function 17 calculates a second trend score Sim_{abnor} between the blood pressure and the body temperature of the patient and the blood pressure and the body temperature of the similar patient in a diseased state.

In this manner, some measured values are excluded and closeness (score) of trend between the patient and a similar patient is calculated in the present embodiment. In this sense, closeness of trend is calculated after a feature amount for which a measurement error has occurred is excluded. As a result, in the present embodiment, it is possible to classify classes having measurement errors and diseases (because information on measurement errors is not used). Since biometric information in which measurement error has occurred is not known, exhaustive searching is performed while excluding pieces of biometric information one by one and calculating a trend score in the present embodiment. FIG. 4 and FIG. 5 show a case in which a measurement error has occurred in one piece of the biometric information. The number of measurement errors may be determined depending on the type of patient information. Although an example in which there are three pieces of biometric information in the examples shown in FIG. 4 and FIG. 5, the number of pieces of biometric information may be, for example, 2, 4 or more.

The trend score increases as trends of patient biometric information and measured values of a similar patient become closer, that is, as measured values become closer, and decreases as trends of the patient biometric information and the measured values of the similar patient becomes further from each other, that is, as measured values separate from each other. An example of the trend score calculation method will be described later.

In the examples described using FIG. 4 and FIG. 5, there is a single condition of the similar patient. In the following example, an example of a trend score calculation method when a plurality of conditions of a similar patient are extracted from the patient DB 11 will be described with reference to FIG. 6. FIG. 6 is a diagram showing a trend score calculation method when a plurality of conditions of a similar patient are extracted from the patient DB. Examples in which a plurality of healthy states are present include, for example, a case in which a chronic disease has not developed, a case in which a chronic disease develops, different measured values depending on time periods (for example, morning and night), and the like. Examples in which a plurality of conditions at the time of disease onset are present include, for example, early disease onset, middle disease onset, and the like.

Even in the same healthy/diseased state, there is a possibility that trend of a trend value differs depending on condition. Therefore, in order to handle such cases, a trend score may be calculated for each condition as described above in the present embodiment. In such a case, the first calculation function 16 and the second calculation function 17 calculate a trend score for each condition. For example, as shown in FIG. 6, the first calculation function 16 calculates a first trend score Simₙₒᵣ between patient biometric information and measured values of a similar patient in a healthy state in a first condition nor_0 and calculates a first trend score Simₙₒᵣ between the patent biometric information and measured values of the similar patient in the healthy state in a second condition nor_1 after excluding the blood pressure. The second calculation function 17 calculates a second trend score Sim_{abnor} between the patient biometric information and measured values of the similar patient in a diseased state in the first condition nor_0 and calculates a second trend score Sim_{abnor} between the patent biometric information and measured values of the similar patient in the diseased state in the second condition nor_1 after excluding the blood pressure.

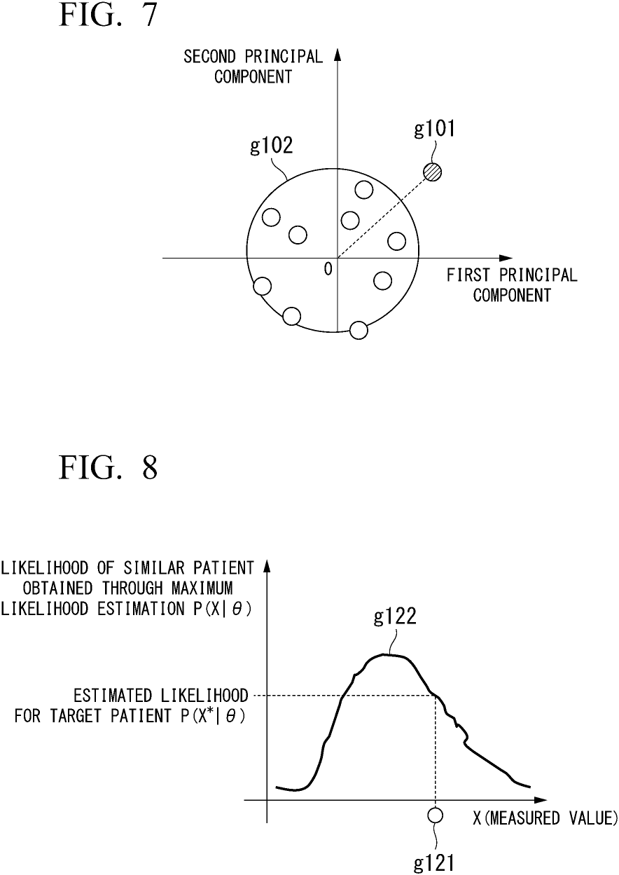

Next, an example of a method of obtaining a trend score value will be described using FIG. 7 and FIG. 8. FIG. 7 is a diagram showing a method of obtaining a trend score according to principal component analysis. A first calculation method is a method using a method of principal component analysis. In the example of FIG. 7, a plurality of similar patients to the patient have been extracted from the patient DB 11. Principal components are a blood pressure, a body temperature, a pulse, and the like. Principal component analysis is performed on the remaining measured values after excluding one measured value, as described above. In such a case, the first calculation function 16 and the second calculation function 17 calculate trend scores by analyzing patient biometric information and measured values of the similar patients through the principal component analysis method and calculating a distance between the central position (origin) of a distribution of the similar patients and the position of the patient. The first calculation function 16 and the second calculation function 17 calculate trend scores, for example, by calculating the reciprocal of T² statistic (the square of the distance from the origin in principal component analysis) as in the following formula (1). In formula (1), i is the type of biometric information (for example, blood pressure i=0, body temperature i=1, pulse i=2).

$${T}^{2} \text{statistic} = {Σ}_{i = 0}^{m} \frac{{t}_{i}}{{σ}_{i}}$$

In a second calculation method, trend scores are calculated using a maximum likelihood estimation method. FIG. 8 is a diagram showing a method of obtaining a trend score according to maximum likelihood estimation. In this case, each of the first calculation function 16 and the second calculation function 17 calculate trend scores by calculating a likelihood (P(X*|Θ)) of a target patient with respect to a parameter (Θ) obtained by performing maximum likelihood estimation (P(X|Θ)) on a similar patient, as shown in FIG. 8. For example, in FIG. 8, point g121 represents patient biometric information, and line g122 represents measured values of the similar patient. The first calculation function 16 calculates a first trend score by calculating a likelihood of the target patient with respect to a parameter obtained by performing maximum likelihood estimation on the similar patient in a healthy state. The second calculation function 17 calculates a second trend score by calculating a likelihood of the target patient with respect to a parameters obtained by performing maximum likelihood estimation on the similar patient in a diseased state.

The first calculation function 16 and the second calculation function 17 may obtain a measured value distribution and a probability distribution for each similar patient in FIG. 7 and FIG. 8 when there is a large number of measured values of one similar patient. Alternatively, the first calculation function 16 and the second calculation function 17 may obtain a measured value distribution and a probability distribution of a similar patient using measured values of a plurality of similar patients. In addition, in the first calculation method or the second calculation method, the first calculation function 16 and the second calculation function 17 calculate trend scores using biometric information obtained with respect to a target patient. For this reason, the biometric information of the target patient is, for example, a measured value measured at a predetermined timing (for example, in the morning) among data acquired in chronological order every predetermined time.

The trend score calculation methods described using FIG. 7 and FIG. 8 are examples, and are not limited thereto. The first calculation function 16 and the second calculation function 17 may calculate trend scores using, for example, other statistical methods.

### Processing Performed by Determination Function

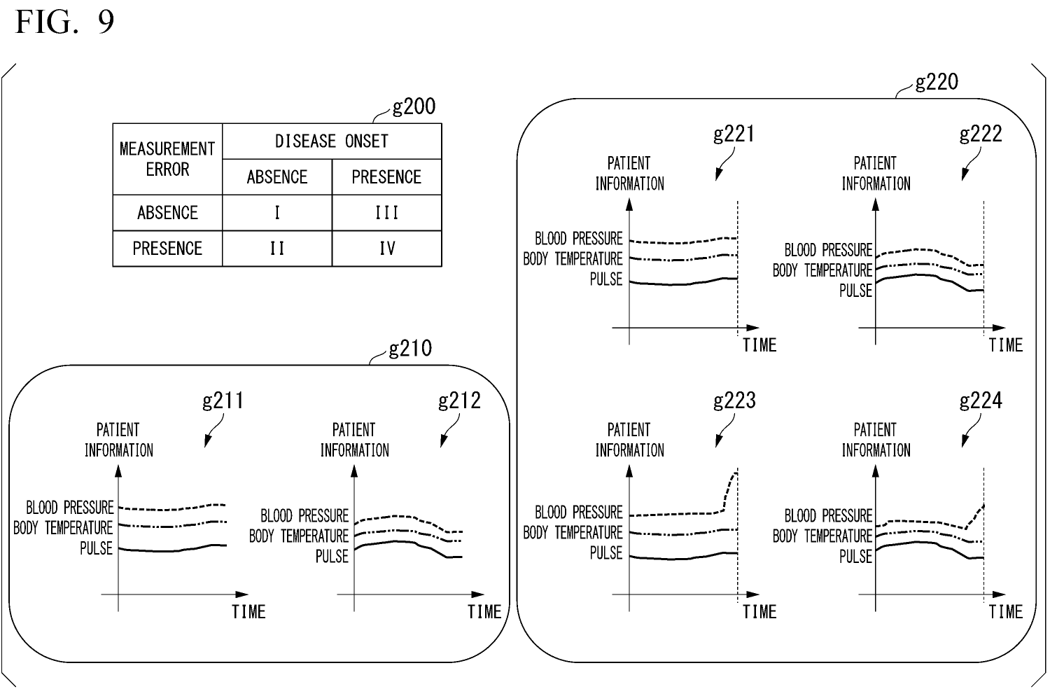

Next, processing performed by the determination function 18 will be described. FIG. 9 is a diagram showing processing performed by the determination function. Table g200 shows cases assumed for patient biometric information. As shown in Table g200, case I having no measurement error and no disease onset, case II having a measurement error and no disease onset, case III having no measurement error and a disease onset, case IV having a measurement error and a disease onset are assumed. Temporal change in the patient biometric information shown in FIG. 9 is an image in each case, and there is a single trend score or measured value used for comparison which is not temporal, for example.

Graphs g211 and g212 surrounded by square g210 represent examples of measured values of a similar patient. Graph g211 shows measured values of the similar patient in a healthy state, and graph g212 shows measured values of the similar patient at the time of disease onset. In graphs g211 and g212, the horizontal axis represents time and the vertical axis represents measured values (blood pressure, body temperature, and pulse).

Graphs g221 to g224 surrounded by square g220 represent examples of patient biometric information for each case. Graph g221 shows patient biometric information for case I, graph g222 shows patient biometric information for case II, graph g223 shows patient biometric information for case III, and graph g224 shows patient biometric information for case IV. In graphs g221 to g224, the horizontal axis represents time and the vertical axis represents measured values (blood pressure, body temperature, and pulse).

The determination function 18 compares calculated trend scores to estimate presence or absence of a disease onset and a measurement error. In this case, the determination function 18 estimates which case in table g200 is a corresponding case by assuming a case with a high possibility of corresponding among the assumed cases described above.

For example, in case I, that is, in a case having no measurement error and no disease onset, it is assumed that patient biometric information is close to measured values of a similar patient in a healthy state. In case III, that is, in a case having no measurement error and a disease onset, it is assumed that the patient biometric information is close to measured values of the similar patient in a diseased state. In case II, that is, in a case having a measurement error and no disease onset, it is assumed that the patient biometric information is close to the measured values of the similar patient in the healthy state, but the patient biometric information has changed or is different. In case IV, that is, in a case having a measurement error and a disease onset, the patient biometric information is close to the measured values of the similar patient in the diseased state, but the patient biometric information has changed or is different.

For this reason, in the present embodiment, the determination function 18 classifies the aforementioned cases on the basis of a size relationship between the first trend score between the patient biometric information and the measured values of the similar patient in the healthy state, and the second trend score between the patient biometric information and the measured values of the similar patient in the diseased state, and a size relationship between the difference (or ratio) and a threshold value.

FIG. 10 is a flowchart showing an example of a trend score calculation and determination processing procedure. The first calculation function 16 and the second calculation function 17 initialize n (for example, 0) (step S101). The first calculation function 16 and the second calculation function 17 exclude an n-th measured value and calculate trend scores (step S102). The first calculation function 16 and the second calculation function 17 determine whether or not n is equal to m (step S102). If n is not equal to m, the first calculation function 16 and the second calculation function 17 add 1 to n (step S104) and returns to step S102.

If n is equal to m, the determination function 18 acquires a first trend score and a second trend score (step S105). The determination function 18 compares the first trend score with the second trend score (step S106). The determination function 18 classifies cases into three cases, i.e., case I, case II, and case III or case IV on the basis of the result of comparison (step S107).

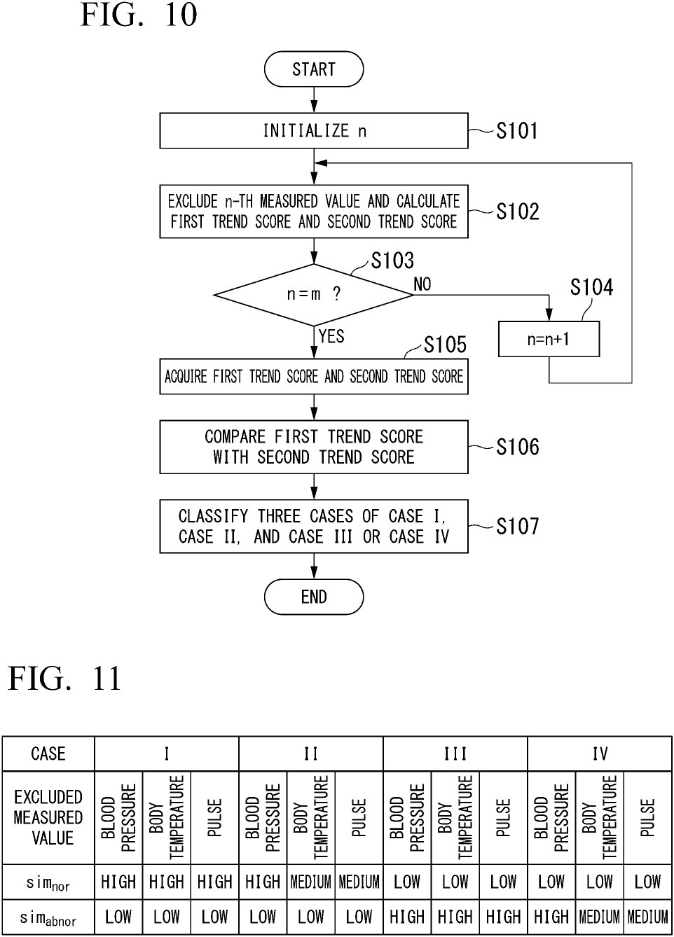

FIG. 11 is a diagram showing examples of the first trend score and an example of the second trend score for each case. In FIG. 11, a high trend score is a normalized score of, for example, 0.8 or more, a medium trend score is a normalized score of, for example, 0.7 to 0.4, and a low trend score is normalized score of, for example, 0.3 or less. In the example of FIG. 11, case I corresponds to a case in which the first trend score Simₙₒᵣ is high and the second trend score Sim_{abnor} is low when the blood pressure has been excluded, the first trend score Simₙₒᵣ is high and the second trend score Sim_{abnor} is low when the body temperature has been excluded, and the first trend score Simₙₒᵣ is high and the second trend score Sim_{abmpr}is low when the pulse has been excluded.

In addition, case II corresponds to a case in which the first trend score Simₙₒᵣ is high and the second trend score Sim_{abnor} is low when the blood pressure has been excluded, the first trend score Simₙₒᵣ is medium and the second trend score Sim_{abnor} is low when the body temperature has been excluded, and the first trend score Simₙₒᵣ is medium and the second trend score Sim_{abnor} is low when the pulse has been excluded.

Further, case III corresponds to a case in which the first trend score Simₙₒᵣ is low and the second trend score Sim_{abnor} is high when the blood pressure has been excluded, the first trend score Simₙₒᵣ is low and the second trend score Sini_{abnor} is high when the body temperature has been excluded, and the first trend score Simₙₒᵣ is low and the second trend score Sim_{abnor} is high when the pulse has been excluded.

In addition, case IV corresponds to a case in which the first trend score Simₙₒᵣ is low and the second trend score Sim_{abnor} is high when the blood pressure has been excluded, the first trend score Simₙₒᵣis low and the second trend score Sim_{abnor} is medium when the body temperature has been excluded, and the first trend score Simₙₒᵣ is low and the second trend score Sim_{abnor} is medium when the pulse has been excluded.

In the present embodiment, the determination function 18 classifies cases into three cases: case I; case II; and case III or case IV on the basis of trends shown in FIG. 11. The example of temporal changes in the measured values shown in FIG. 9 and the example of high/medium/low relationships of trend scores for each case shown in FIG. 11 are examples, and the present invention is not limited thereto.

### Example of Processing of Medical Information Processing Device

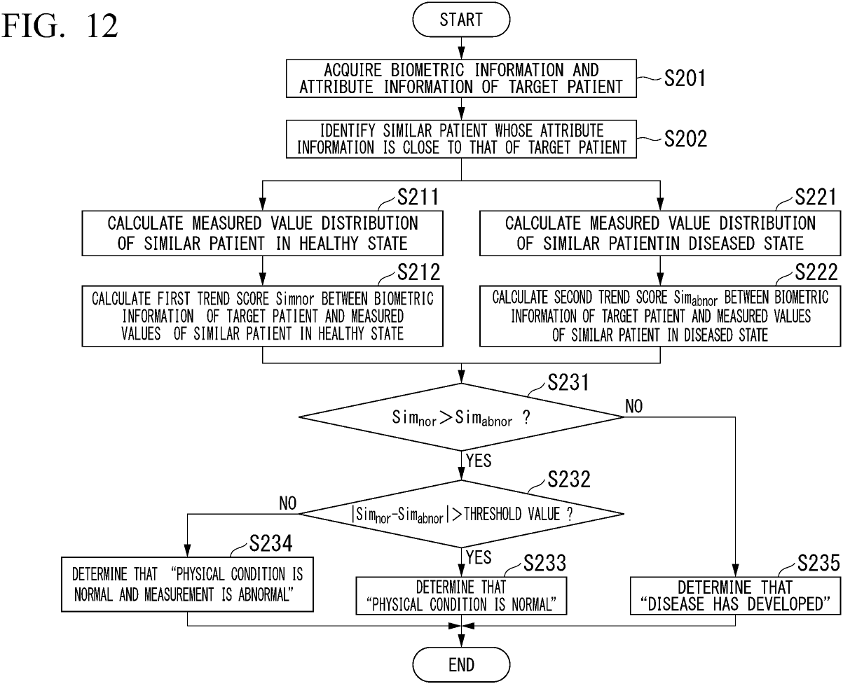

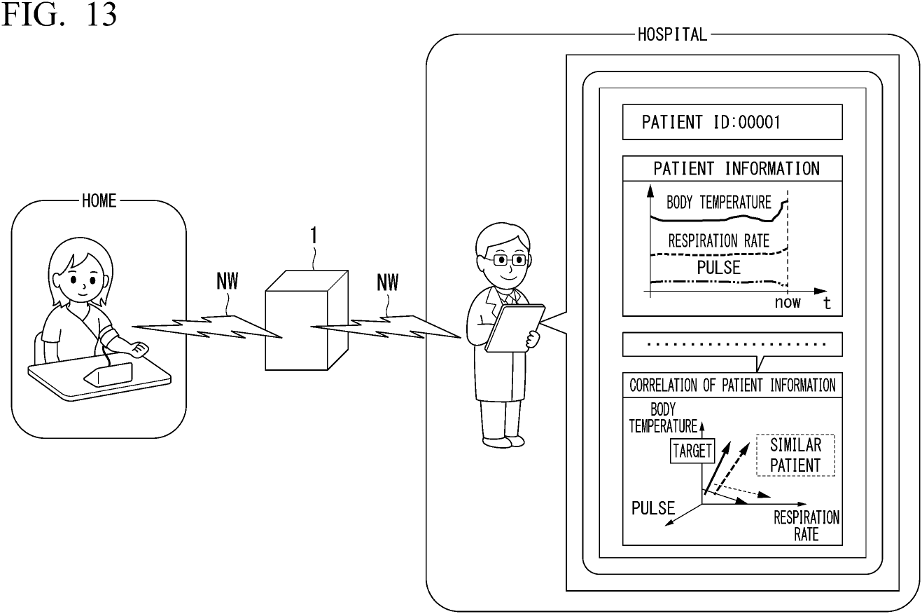

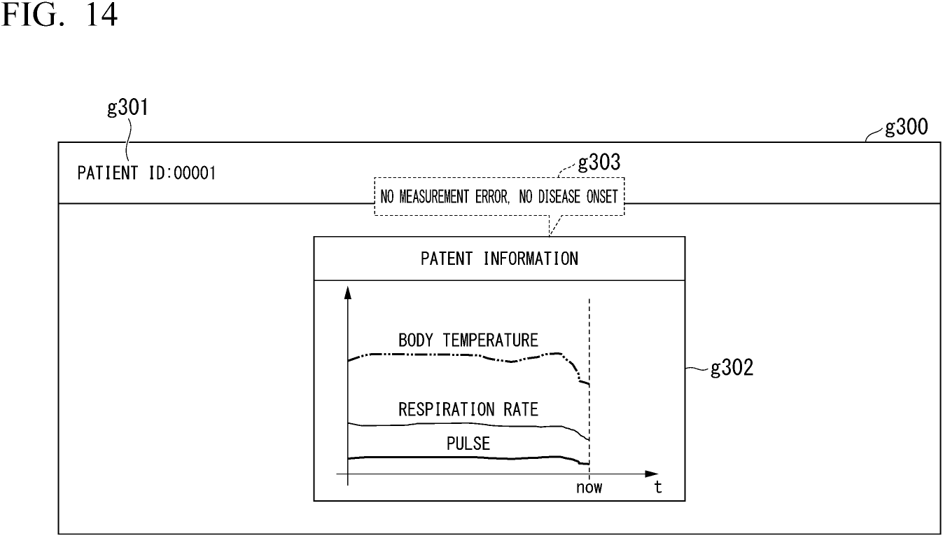

Next, an example of a processing procedure performed by the medical information processing device 1 will be described. FIG. 12 is a flowchart of an example of a processing procedure performed by the medical information processing device according to the present embodiment.

The acquisition function 13 acquires patient information (biometric information and attribute information) of a patient (step S201).

The identification function 14 identifies a similar patient whose attribute information is similar to that of the patient from the patient DB 11 on the basis of the acquired attribute information of the patient (step S202).

The identification function 14 calculates a measured value distribution of the similar patient in a healthy state using patient information on the identified similar patient (step S211). The first calculation function 16 calculates a first trend score Simₙₒᵣ between patient biometric information and the measured value distribution of the similar patient in the healthy state (step S212).

The identification function 14 calculates a measured value distribution of the similar patient in a diseased state (step S221). The second calculation function 17 calculates a second trend score Sim_{abnor} between the patient biometric information and the measured value distribution of the similar patient in the diseased state (step S222).

The determination function 18 determines whether or not the first trend score Simₙₒᵣ is greater than the second trend score Sim_{abnor}(step S231). If the first trend score Simₙₒᵣ is greater than the second trend score Sim_{abnor}, the determination function 18 determines whether or not the absolute value of the difference between the first trend score Simₙₒᵣ and the second trend score Sim_{abnor} is greater than a threshold value (step S232).

If the first trend score Simₙₒᵣ is greater than the second trend score Sim_{abnor} and the absolute value of the difference between the first trend score Simₙₒᵣ and the second trend score Sim_{abnor} is greater than the threshold value, the determination function 18 determines that “physical condition is normal” because there is no measurement error and no disease onset in measured values of the subject patient (case I) (step S233).

If the first trend score Simₙₒᵣ is greater than the second trend score Sim_{abnor}and the absolute value of the difference between the first trend score Simₙₒᵣand the second trend score Sim_{abnor} is equal to or less than the threshold value, the determination function 18 determines that “physical condition is normal and measurement is abnormal” because there is a measurement error and no disease onset in the measured values of the target patient (case II) (step S234).

If the second trend score Sim_{abnor} is equal to or greater than the first trend score Simₙₒᵣ, the determination function 18 determines that “a disease has developed” because there is a disease onset (case III or IV) (step S235).

The reason why the determination function 18 determines that a disease has developed regardless of whether there is a measurement error is that it is important to present to a user (for example, a doctor, a nurse, a caregiver, or the like) that a disease has developed.

The processing procedure shown in FIG. 12 is an example, and is not limited thereto. For example, the medical information processing device 1 may perform processing of steps S211 and S212 and processing of steps S221 and S222 at the same time or through time-division processing, or may perform processing of steps S211 and S212 after processing of steps S221 and S222.

### Example

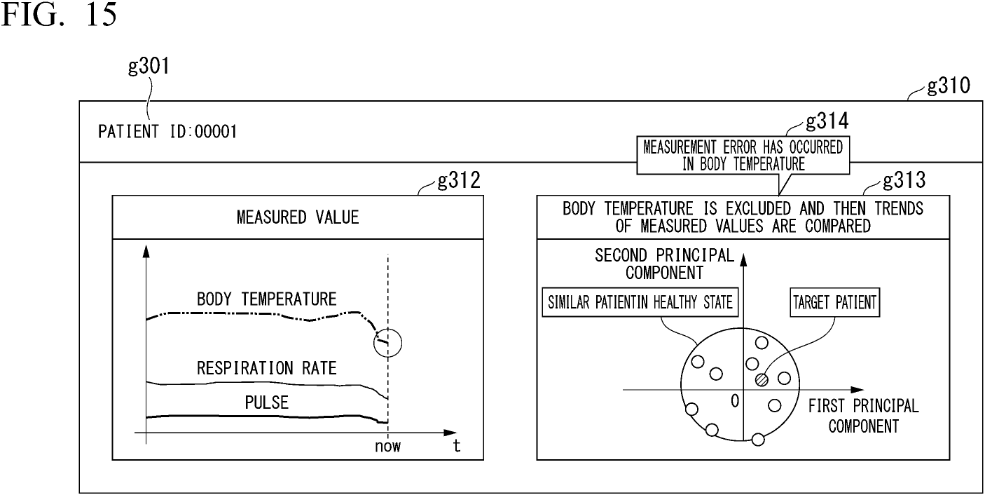

Next, an example of a usage scenario of the medical information processing device 1 will be described. FIG. 13 is a diagram showing an example of a usage scenario of the medical information processing device. In the following example, the medical information processing device 1 determines presence or absence of a measurement error or a disease onset on the basis of vital signs measured by a home patient (target patient) and shares the results with a doctor in a hospital as shown in FIG. 13. A measuring apparatus used for measurement by the target patient at home or the like may have, for example, a wireless communication function, and may transmit biometric information to the medical information processing device 1 via a network NW. Alternatively, the target patient, family of the target patient, or the like may input the biometric information to a terminal (e.g., a personal computer, a tablet terminal, a smartphone, or the like) at the target patient’s home such that the terminal transmits it to the medical information processing device 1 via the network NW. Alternatively, the terminal may perform proximity communication with the measuring apparatus to acquire biometric information and transmit the acquired biometric information to the medical information processing device 1 via the network NW. The measuring apparatus may be, for example, a wearable terminal such as a smartwatch having a sensor capable of detecting biometric information and a terminal function.

FIG. 14 is a diagram showing an example of information presented when it is determined that there is no measurement error and no disease onset. FIG. 14 shows an example of an image g300 displayed on an image display device of a personal computer of a tablet terminal used by a doctor, for example, in a case where the medical information processing device determines that biometric information is case I. The image g300 includes, for example, a patient ID (patient identification information) image g301 and an image g302 representing temporal changes in measured values included in patient information. In this case, the medical information processing device 1 may present only patient information as shown in FIG. 14. The medical information processing device 1 may determine that, for example, “there is no measurement error and no disease onset” and present an image g303 showing the determination result.

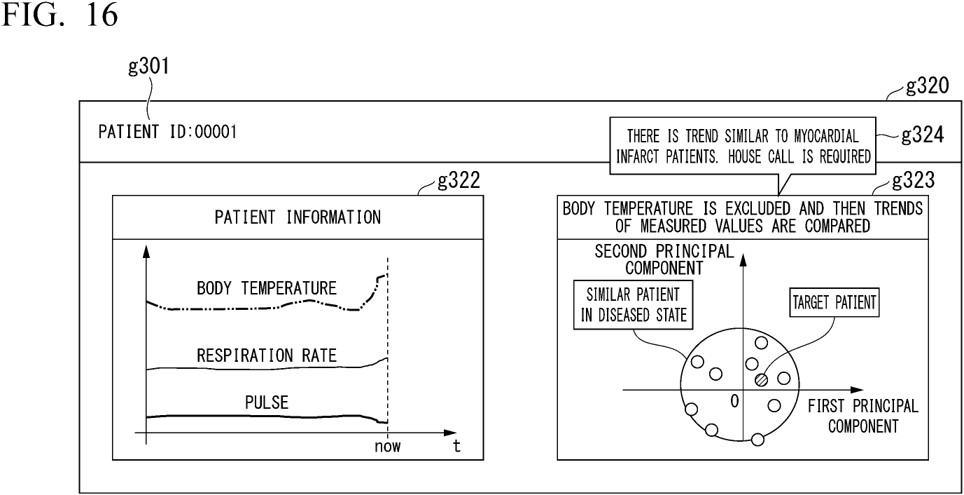

FIG. 15 is a diagram showing an example of information presented when it is determined that there is a measurement error and no disease onset. FIG. 15 shows an example of an image g310 displayed on an image display device of a personal computer or a tablet terminal used by a doctor, for example, in a case where the medical information processing device 1 determines that biometric information is case II. The image g310 includes, for example, a patient ID image g301, an image g312 representing temporal changes in measured values included in patient information, an image g313 representing principal component analysis results, for example, and an image g314 representing a message suggesting a measurement error based on determination results (for example, “measurement error occurs in body temperature”). The medical information processing device 1 may also present an image representing, for example, “there is a measurement error and no disease onset.”

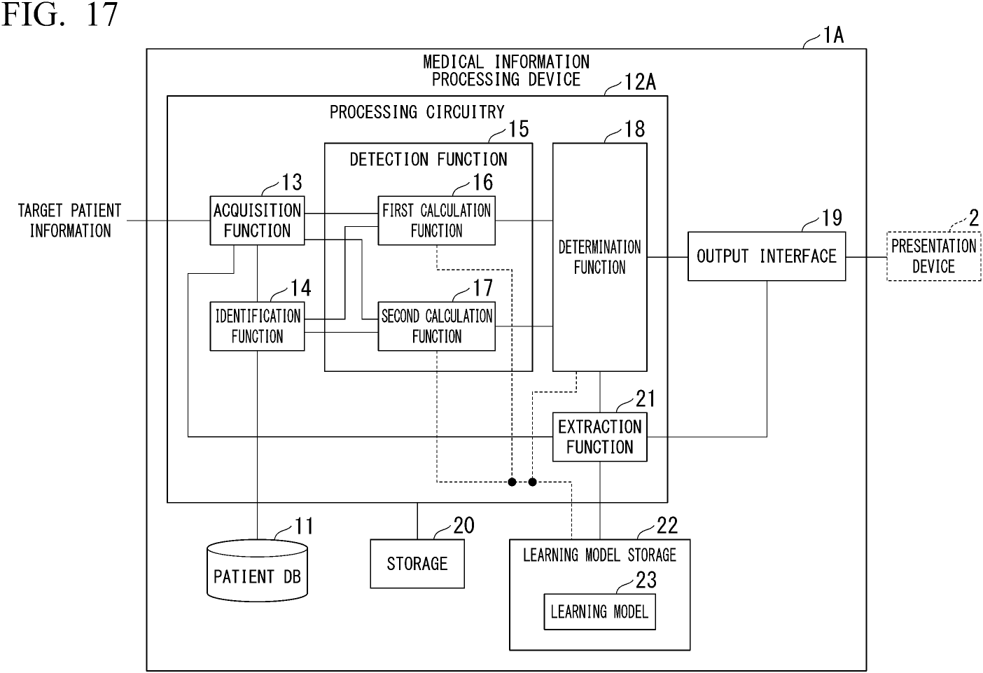

FIG. 16 is a diagram showing an example of information presented when it is determined that a disease has developed. FIG. 16 shows an example of an image g320 displayed on an image display device of a personal computer or a tablet terminal used by a doctor, for example, in a case where the medical information processing device 1 determines that biometric information is case III or IV. The image g320 includes, for example, a patient ID image g301, an image g322 representing temporal changes in measured values included in patient information, an image g323 representing principal component analysis results, for example, and an image g324 representing a message demanding a house call based on determination results (for example, “there is a trend similar to myocardial infarct patients. House call is required.”).

The presented images shown in FIG. 14 to FIG. 16 are examples, and are not limited thereto. The presented images may also include other information (for example, the age, sex, and disease of the patient).

As described above, in the present embodiment, the medical information processing device 1 includes the acquisition function 13 for acquiring biometric information on a patient, the detection function 15 for detecting trend change in biometric information at a predetermined timing on the basis of time-series data regarding the biometric information, and the determination function 18 for determining whether trend change is due to a measurement error in the biometric information or due to change in the patient’s condition.

Therefore, according to the present embodiment, when an abnormality has occurred in biometric information of a target patient, it is possible to clearly distinguish whether the cause of the abnormality is a measurement abnormality or a disease onset.

In addition, the present embodiment further includes the identification function 14 for identifying a similar patient on the basis of patient attribute information, and the determination function 18 determines the trend change as a condition change on the basis of biometric information on the similar patient and the biometric information of the patient at the predetermined timing.

Thus, according to the present embodiment, the acquisition function 13 acquires a plurality of types of biometric information. Then, a trend score is calculated while excluding the acquired plurality of pieces of biometric information, for example, one by one, in the present embodiment.

Therefore, according to the present embodiment, when a measurement error has occurred in one of three pieces of biometric information, for example, it is possible to appropriately determine that it is a measurement error and present it.

Although an example in which the first trend score and the second trend score are calculated while excluding a plurality of pieces of biometric information one by one, for example, and it is determined whether a trend change in the biometric information is due to a measurement error in the biometric information or due to a change the patient’s condition on the basis of the calculated first trend score and the second trend score has been described in the above-described example, the present invention is not limited thereto. There may be one piece of biometric information.

When there is one piece of biometric information, the medical information processing device 1 identifies a similar patient on the basis of attribute information included in patient biometric information. The medical information processing device 1 calculates a first trend score on the basis of the patient biometric information and measured values of a similar patient in a healthy state and calculates a second trend score on the basis of the patient biometric information and measured value of the similar patient in a diseased state. The medical information processing device 1 calculates trend scores using, for example, the maximum likelihood estimation method, which is the second calculation method described above. The medical information processing device 1 may determine whether a trend change in biometric information is due to a measurement error in the biometric information or due to a change in the patient’s condition on the basis of the first trend score and the second trend score.

Even if there is only one piece of biometric information, when an abnormality has occurred in biometric information of a target patient, it is possible to clearly distinguish whether the cause of abnormality is a measurement abnormality or a disease onset according to the present embodiment.

### Modified Example

In the example using FIG. 15 and FIG. 16, an example in which the medical information processing device 1 determines whether biometric information is case I, case II, or case III or IV, and presents the determination result has been described, but the present invention is not limited thereto. For example, the medical information processing device may detect data in which a measurement error has occurred and screen training data as preprocessing before a clinical decision support (CDS) model is caused to read data.

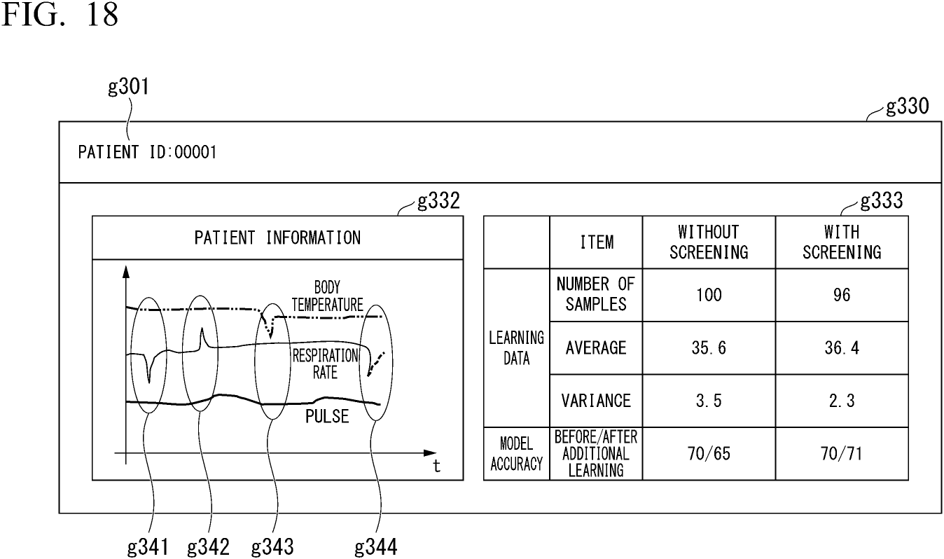

FIG. 17 is a diagram showing an example of a configuration of a medical information processing device of a modified example. As shown in FIG. 17, the medical information processing device 1A includes a patient DB 11, processing circuitry 12A, an output interface 19, a storage 20, and a learning model storage 22. The processing circuitry 12A includes, for example, an acquisition function 13, an identification function 14, a detection function 15, a determination function 18, and an extraction function 21. The detection function 15 includes a first calculation function 16 and a second calculation function 17. The learning model storage 22 includes a learning model 23.

The learning model 23 is, for example, a model for CDS. The CDS model may be one used in medical practice.

The extraction function 21 performs, for example, moving average processing on biometric information acquired by the acquisition function 13 for each sampling time and for each type of biometric information (e.g., body temperature, respiration rate, and pulse) to extract biometric information having a difference of a predetermined value or more from a moving average value and counts the biometric information. The sampling time is, for example, every second.

In addition, the processing circuitry 12A realizes the functions of the acquisition function 13, the identification function 14, the detection function 15, the first calculation function 16, the second calculation function 17, the determination function 18, and the extraction function 21 for example, by a hardware processor executing a program stored in the storage 20.

FIG. 18 is a diagram showing an example of an image presented by a presentation device. An image g330 includes, for example, a patient ID image g301, an image g332 representing temporal changes in biometric information (measured values) included in patient information, and a screening result image g333 with respect to measured values. In the image g332, measured values surrounded by ellipses g341 to g344 are examples of measured values detected by the extraction function 21 as measured values having differences of a predetermined value or more. The biometric information in the example of FIG. 18 may be, for example, measured values acquired at each sampling time. The image g333 includes, for example, a learning data item and a model accuracy item. The learning data item includes, for example, an item and no screening. The item includes presence of screening, the number of samples, an average value, and a variance value. The model accuracy item has a value representing model accuracy before and after additional learning is performed using data without screening and a value representing model accuracy before and after additional learning is performed using data with screening. The image g333 is an example and is not limited thereto, and may include other items and the like.

According to the present embodiment, it is possible to provide a user with information for determining whether or not to use acquired measurement values for learning of the learning model 23 by presenting such an image g330 to the user. The user may select whether to use measured values with screening or measured values without screening for learning of the learning model 23.

The medical information processing device 1A may select whether to use measured values with screening or measured values without screening for learning of the learning model 23 on the basis of at least one of a variance value and a model accuracy, for example. In this case, the medical information processing device 1A may select use of the measurement values without screening, for example, if the variance value is within a threshold value. Alternatively, the medical information processing device 1A may select use of the measured values without screening, for example, if the difference or the ratio between model accuracy values before and after learning is within a threshold value.

The first calculation function 16, the second calculation function 17, and the determination function 18 may perform calculation of trend scores and determination according to comparison of the trend scores using the learning model 23 trained in this manner.

According to at least one embodiment described above, it is possible to distinguish a measurement error from a disease onset on the basis of closeness of trend to other patients by including the processing circuitry 12 (or 12A).

In the above-described embodiment, the acquisition function 13 is an example of an “acquirer,”, the detection function 15 is an example of a “detector,” the determination function 18 is an example of a “determiner,” the identification function 14 is an example of an “identifier,” and the extraction function 21 is an example of an “extractor.”

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing device comprising processing circuitry configured to:
acquire biometric information on a patient;
detect a trend change in the biometric information at a predetermined timing on a basis of time-series data regarding the biometric information; and
determine whether the trend change is due to a measurement error in the biometric information or due to a change in a condition of the patient.

2. The medical information processing device according to claim 1, wherein the processing circuitry is further configured to:
identify a similar patient on a basis of attribute information of the patient; and
determine the trend change as the change in the condition on a basis of biometric information on the similar patient and the biometric information of the patient at the predetermined timing.

3. The medical information processing device according to claim 1, wherein the processing circuitry is further configured to:
acquire a plurality of types of biometric information;
exclude any one of the acquired plurality of types of biometric information; and
detect a trend change in the biometric information at a predetermined timing.

4. The medical information processing device according to claim 1, wherein the processing circuitry is further configured to:
calculate a first trend score on a basis of the biometric information of the patient at the predetermined timing and biometric information of a similar patient in a healthy state;
calculate a second trend score on a basis of the biometric information of the patient at the predetermined timing and biometric information of the similar patient in a diseased state; and
determine the trend change as the change in the condition on a basis of the first trend score and the second trend score.

5. The medical information processing device according to claim 4, wherein the processing circuitry is further configured to:
calculate the first trend score on a basis of results of principal component analysis performed on the biometric information of the patient at the predetermined timing and the biometric information of the similar patient in the healthy state; and
calculate the second trend score on a basis of results of principal component analysis performed on the biometric information of the patient at the predetermined timing and the biometric information of the similar patient in the diseased state.

6. The medical information processing device according to claim 4, wherein the processing circuitry is further configured to:
calculate the first trend score on a basis of an estimated likelihood of the biometric information of the patient at the predetermined timing and an estimated likelihood of the biometric information on the similar patient in the healthy state; and
calculate the second trend score on a basis of the estimated likelihood of the biometric information of the patient at the predetermined timing and an estimated likelihood of the biometric information on the similar patient in the diseased state.

7. The medical information processing device according to claim 1, wherein
the processing circuitry is further configured to extract data in which the measurement error has occurred from the biometric information of the patient, and
the medical information processing device further comprises an output interface configured to present information on the extracted data.

8. A medical information processing method, using a medical information processing device, comprising
acquiring biometric information on a patient;
detecting a trend change in the biometric information at a predetermined timing on a basis of time-series data regarding the biometric information; and
determining whether the trend change is due to a measurement error in the biometric information or due to a change in a condition of the patient.

9. A computer-readable non-transitory storage medium storing a program causing a computer to:
acquire biometric information on a patient;
detect a trend change in the biometric information at a predetermined timing on a basis of time-series data regarding the biometric information; and
determine whether the trend change is due to a measurement error in the biometric information or due to a change in a condition of the patient.
